(19) United States
(12) Patent Application Publication
(10) Pub. No.: US 2024/0165409 A1
(43) Pub. Date: May 23, 2024

(54) NEURAL STIMULATION AND SENSING SYSTEM FOR AUDITORY AND NON-AUDITORY ACTIVATION

(71) Applicants: BLACKROCK MICROSYSTEMS, INC., Salt Lake City, UT (US); REGENTS OF THE UNIVERSITY OF MINNESOTA, Minneapolis, MN (US); MEDIZINISCHE HOCHSCHULE HANNOVER, Niedersachen (DE); INTERNATIONAL NEUROSCIENCE INSTITUTE, Hannover (DE); MED-EL ELEKTROMEDIZINISCHE GERAATE GESELLSCHAFT m.b.H., Innsbruck (AT); UNIVERSITY OF UTAH, Salt Lake City, UT (US); FEINSTEIN INSTITUTES FOR MEDICAL RESEARCH, Manhasset, NY (US)

(72) Inventors: Moritz Michael Leber, Salt Lake City, UT (US); Robert Kyle Franklin, IV, Centerville, UT (US); Sandeep Negi, Salt Lake City, UT (US); Joseph David Crew, Salt Lake City, UT (US); Janet Liu, Salt Lake City, UT (US); Vinh Quang Ngo, Sandy, UT (US); Hubert Hyoungil Lim, St. Paul, MN (US); Geoffrey Mohon Ghose, Minnetonka, MN (US); Luke Aaron Johnson, St. Paul, MN (US); Inderbir Singh Sondh, Minneapolis, MN (US); Abigail Paige Heiller, Lakeville, MN (US); Meredith Evelyn Adams, Minneapolis, MN (US); Andrew John Oxenham, St. Paul, MN (US); Thomas Heinrich Robert Lenarz, Hannover (DE); Karl-Heinz Hiro Dyballa, Sehnde (DE); Waldo Nogueira Vazquez, Hannover (DE); Amir Samii, Hannover (DE); Keno H. B. Hübner, Innsbruck (AT); Paul Pontiller, Innsbruck (AT); Marco Eder, Mutters (AT); Daniel M. Sieber, Innsbruck (AT); Alexander Mayr, Absam (AT); Guntram Wyzisk, Innsbruck (AT); Elisabeth A. Hansen, Innsbruck (AT); Dominik Hammerer, Innsbruck (AT); Florian Solzbacher, Salt Lake City, UT (US); Loren Wellington Rieth, Morgantown, WV (US)

(21) Appl. No.: 18/469,456

(22) Filed: Sep. 18, 2023

Related U.S. Application Data
(60) Provisional application No. 63/426,551, filed on Nov. 18, 2022.

(51) Int. Cl. A61N 1/36; A61N 1/05

(52) U.S. Cl. CPC A61N 1/3606; A61N 1/0558

(57) ABSTRACT

The present disclosure generally relates to auditory nerve stimulation to create the perception of sound in the brain of a subject such as an animal or human being. In one form, a system includes an implantable electrode array including a plurality of spaced apart micro-needles. The system also includes a first electrical lead electrically coupled to and extending from the implantable electrode array, and an auditory signal device configured to produce one or more electrical signals representative of communications received from an external processor. An interposer is configured to electrically couple the implantable electrode array and the auditory signal device in an arrangement where one or more electrical signals produced by the auditory signal device may be transmitted through the first electrical lead to the implantable electrode array. Various novel stimulation strategies can be employed, such as place modulated stimulation signals.

### RELATED APPLICATIONS

The present application claims priority to U.S. Provisional Patent Application Ser. No. 63/426,551, filed on Nov. 18, 2022 and which is incorporated herein in its entirety by reference.

### STATEMENT OF FEDERALLY FUNDED RESEARCH

This invention was made with government support under contract no. UG3NS107688 awarded by the National Institutes of Health. The government has certain rights in the invention.

### TECHNICAL FIELD

The present disclosure relates to medical implant devices, such as brain implants.

### BACKGROUND

Advances in medical science have enabled sensing and/or modulating of neurological tissue, including the auditory nerve. These advances have allowed scientists and researchers to observe systems like the auditory system in great detail. However, there exists a need in the art for more sophisticated neurological implant devices capable of performing more complex interactions with neurological tissue. Thus, a need exists for a brain implant design that improves capabilities to interact with neurological tissue (e.g., the auditory nerve), and that can do so reliably.

### SUMMARY

The present disclosure generally relates to neuroscience, and more particularly but not exclusively, to auditory nerve stimulation to create the perception of sound in the brain of a subject such as an animal or human being. In one aspect, an implantable electrode array including a plurality of spaced apart micro-needles may be implanted into the auditory nerve bundle of an animal or human being and stimulate the auditory nerve in response to receiving electrical signals representative of observed sound(s).

Hearing impaired individuals may benefit from the use of hearing aids or cochlear implants. However, there remains a need for further contributions in this area of technology, particularly in advancements in auditory nerve implants that are capable of bypassing the ear structure entirely.

The claimed subject matter is not limited to embodiments that solve any disadvantages or that operate only in environments such as those described above. Rather, this background is only provided to illustrate examples of where the present disclosure may be utilized.

In one embodiment, a system includes an implantable electrode array including a plurality of spaced apart micro-needles. The system also includes a first electrical lead electrically coupled to and extending from the implantable electrode array, and an auditory signal device configured to produce one or more electrical signals representative of observed sound. An interposer is configured to electrically couple the implantable electrode array and the auditory signal device in an arrangement where one or more electrical signals produced by the auditory signal device may be transmitted through the first electrical lead to the implantable electrode array.

In another embodiment, an apparatus includes an implantable electrode array including a plurality of spaced apart micro-needles, an electrical lead electrically coupled to and extending from the implantable electrode array and including a plurality of wires, and an interposer including a number of contact pads. One or more of the plurality of wires of the electrical lead may be individually and electrically coupled to respective ones of the number of contact pads.

Some embodiments described herein can include three-dimensional electrode structures. Examples of such systems are described in U.S. Pat. No. 5,215,088 (“the '088 patent”), filed Nov. 7, 1989 and entitled, “Three-Dimensional Electrode Device,” the disclosure of which is hereby incorporated by reference in its entirety. Some embodiments described herein can interact with an already implanted three-dimensional electrode structure, such as those described in the '088 patent.

In still another embodiment, a method includes providing a system including:
an implantable electrode array including a plurality of spaced apart micro-needles;
a first electrical lead electrically coupled to and extending from the implantable electrode array;
an auditory signal device configured to produce one or more electrical signals representative of observed sound; and
an interposer configured to electrically couple the implantable electrode array and the auditory signal device in an arrangement where one or more electrical signals produced by the auditory signal device may be transmitted through the first electrical lead to the implantable electrode array.

The method also includes implanting the implantable electrode array in an auditory nerve of a human being, and positioning the auditory signal device at a discrete location on the human being spaced from the implantable electrode array.

In alternative embodiments, assemblies, systems, apparatuses, and devices relating to auditory nerve stimulation are provided.

Some embodiments of the apparatuses and methods described herein can be used for in-vivo implementations, i.e., to interface with a neural system of a live organism. Some embodiments of the apparatuses and methods described herein can be used to interface with a neural system of a live organism that is awake and performing a behavior.

This Summary is provided to introduce a selection of concepts in a simplified form that are further described below in the Detailed Description. This Summary is not intended to identify key features or essential characteristics of the disclosed subject matter, nor is it intended to be used as an aid in determining the scope of the disclosed subject matter.

Additional features and advantages will be set forth in the description which follows, and in part will be obvious from the description, or may be learned by the practice of the invention. The features and advantages may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

FIG. 1 is a schematic illustration of a system configured for auditory nerve stimulation.

FIG. 2 is a side view of an implantable electrode array of the system of FIG. 1.

FIG. 3 is a perspective view of a portion of the implantable electrode array of FIG. 2 including a plurality of micro-needles.

FIG. 4 is a side view of the portion of the implantable electrode array illustrated in FIG. 3.

FIG. 5 is an enlarged photographic image of the implantable electrode array of the system of FIG. 1 with an electrical lead extending therefrom.

FIG. 6 is a schematic illustration of electrical coupling between components of the system of FIG. 1 provided by an interposer.

FIG. 7 is a plan view of an instrument configured to deliver and implant the implantable electrode array of the system of FIG. 1.

FIGS. 8A-C are perspective views of an alternative versions of end portion of the instrument of FIG. 7.

FIGS. 9A-9B illustrate example experimental results of stimulation responses.

### DETAILED DESCRIPTION

For purposes of promoting an understanding of the present disclosure, reference will now be made to the following embodiments and specific language will be used to describe the same. It will nevertheless be understood that no limitation of the scope of the disclosure is thereby intended, such alterations and further modifications in the described subject matter, and such further applications of the disclosed principles as described herein being contemplated as would normally occur to one skilled in the art to which the disclosure relates.

The present disclosure generally relates to neuroscience, and more particularly but not exclusively, to auditory nerve stimulation to create the perception of sound in the brain of a subject such as an animal or human being. In one aspect, an implantable electrode array including a plurality of spaced apart micro-needles may be implanted into the auditory nerve bundle and stimulate the auditory nerve in response to receiving electrical signals representative of observed sound(s).

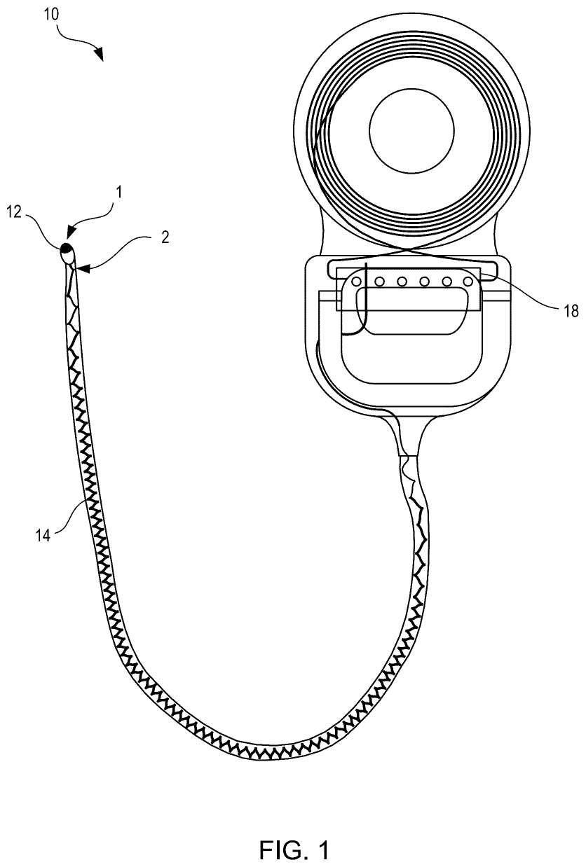

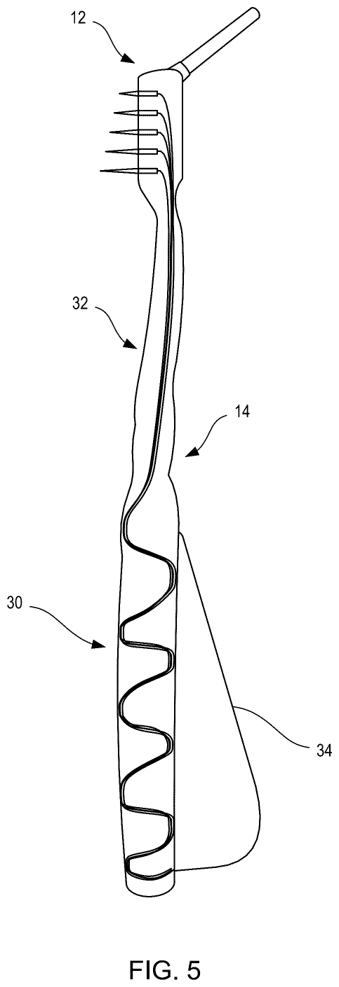

With reference now to FIGS. 1-5 for example, a system 10 includes an implantable electrode array 12, an electrical lead 14 electrically coupled to and extending from the implantable electrode array 12, and an auditory signal device 16 configured to produce one or more electrical signals representative of observed sound. The system 10 further includes an interposer 18 configured to electrically couple the implantable electrode array 12 and the auditory signal device 16 in an arrangement where one or more electrical signals produced by the auditory signal device 16 may be transmitted through the electrical lead 14 to the implantable electrode array 12. Thus, the auditory signal device 16, the interposer 18, the electrical lead 14, and the implantable electrode array 12 are all in electrical communication with each other.

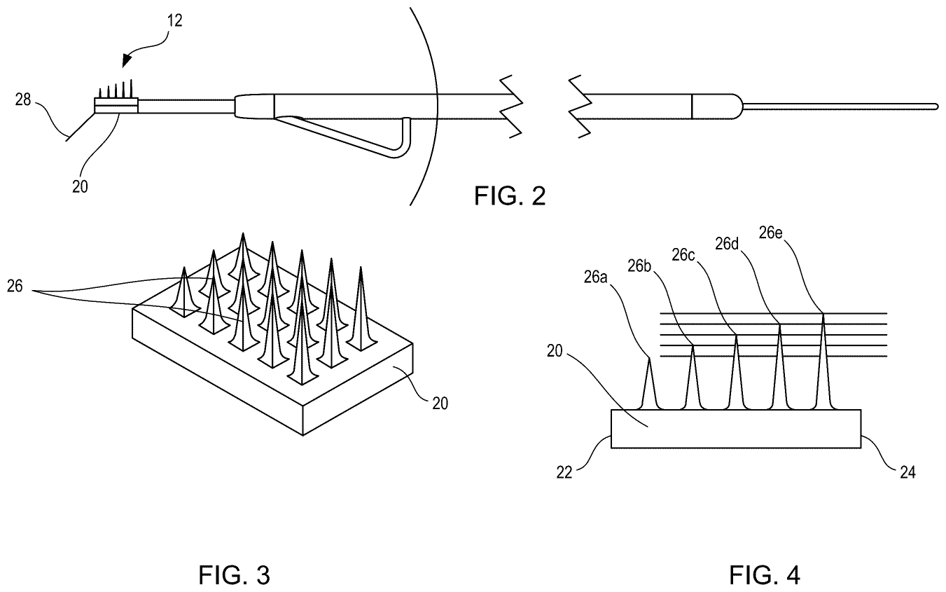

Further details of the implantable electrode array 12 and the electrical lead 14 are illustrated in FIGS. 2-5. More specifically, the implantable electrode array 12 includes a base portion 20 which extends between a first end 22 and an opposite second end 24. The base portion 20 includes a plurality of micro-needles 26 extending therefrom. Generally speaking, the micro-needles 26 may be configured to be positioned in a nerve or nerve bundle, such as an auditory nerve or auditory nerve bundle. In the illustrated form, the implantable electrode array 12 includes 15 micro-needles 26, although other variations in the number of the micro-needles 26 present are possible. The micro-needles 26 are arranged in a grid defined by five rows with each including three of the micro-needles 26. Variations in the arrangement and layout of the micro-needles 26 are possible and contemplated.

As best illustrated in FIG. 4 for example, the micro-needles 26 in each row include a length which is different from the length of the micro-needles 26 in each of the other rows. More specifically, the micro-needles 26a closest to the first end 22 of the base portion 20 can have the shortest length, and the length of the micro-needle 26 can increase in each row in the direction of the second end 24. For example, the micro-needles 26b are taller than the micro-needles 26a, the micro-needles 26c are taller than the micro-needles 26b, the micro-needles 26d are taller than the micro-needles 26c, and the micro-needles 26e are taller than the micro-needles 26d. In this arrangement, a plane that extends along the tips of the micro-needles 26 extends transversely to a plane extending between the first end 22 and the second end 24 of the base portion 20.

In some alternative implementations, the micro-needles 26 comprise anchoring shanks or micro-needles. Such anchoring shanks can be sized similar to (or the same as) the longest shank in the implantable electrode array 12. Additionally, anchoring shanks can be positioned at opposite rows relative to the rows of the longest shanks. In other terms, anchoring shanks can be positioned at opposite ends of the implantable electrode array 12 than the longest shank. Thus, in an alternative example to FIG. 4, the micro-needle 26a would be an anchoring shank if the micro-needle 26a were sized in length to match the micro-needle 26e (the longest shank).

In these or other examples, the micro-needles 26 (e.g., electrode shanks) can include a variety of different lengths. In at least some examples, the shank lengths range from about 0.1 mm to about 1.2 mm, thereby capable of extending across an entire cross-sectional area of the auditory nerve for stimulation. In particular implementations, the shank lengths are about 0.9 mm (e.g., when averaged). As used herein, the term “about” in reference to shank length should be interpreted as +/− (plus or minus) 10% of a value. Other examples within the scope of this disclosure can include different shank lengths.

In certain configurations, one or more shanks in a larger shank-configuration can be deactivated to ensure a nerve is fully spanned by the implantable electrode array 12. For instance, the outer two shortest shanks can be deactivated (leaving the middle shortest shank in the row active). Additionally or alternatively, one of the longest outer shanks can also be deactivated. Thus, for an example 3×5 pin configuration, 12 or 13 of the pins may be active, and the remaining may be deactivated (e.g., for a successful and reliable implantation and signal transmission). This approach may, in some instances, be more advantageous than smaller shank configurations in which all the electrodes are kept active (e.g., because one or more shorter shanks may not be positioned deep enough, one or more longer shanks may be positioned too close to the opposing nerve surface, etc.).

As suggested above, the implantable electrode array 12 can also include various configurations of the micro-needles 26. For example, in some embodiments, the micro-needles 26 comprise a 3×5 (e.g., a three-row by five-column or 15-pin) arrangement of the micro-needles 26. As another example, the micro-needles 26 comprise a 3×4 (e.g., a three-row by four-column or 12-pin) arrangement of the micro-needles 26. Still, in other examples, the micro-needles 26 comprise a 6×9 (e.g., a six-row by nine-column or 54-pin) arrangement of the micro-needles 26. Further, in some examples, the micro-needles 26 comprise a 6×10 (e.g., a six-row by 10-column or 60-pin) arrangement of the micro-needles 26.

In these or other embodiments, the micro-needles 26 can have various spacing or pitch between each micro-needle. For instance, the 3×5 arrangement of the micro-needles 26 can include an electrode pitch of 400 μm. As another example, the 6×9 or 6×10 arrangements of the micro-needles 26 can include an electrode pitch of 200 μm.

In some embodiments, the micro-needles 26 of the implantable electrode array 12 can be positioned along a nerve based on the nerve location and/or the nerve direction. For example, in some implementations, the longest shanks of the micro-needles 26 can be positioned closer to the cochlear side of the auditory nerve, and the shortest shanks of the micro-needles 26 can be positioned closer to the brainstem side of the auditory nerve. In particular embodiments, this example configuration enables the electrical lead 14 to project away from the shorter shanks of the implantable electrode array 12 and towards bony grooves (e.g., for anchoring the electrical lead 14).

In one form, the implantable electrode array 12 may be formed from a wafer including a wafer surface which has been shaped to correspond to the plane that extends along the tips of the micro-needles 26. A plurality of trenches may be cut into the wafer to form a plurality of columns having tops that align with the plane that extends along the tips of the micro-needles 26. The columns may be rounded and sharpened by etching to form the micro-needles 26. Further details in connection with the formation of the implantable electrode array 12 may be found in U.S. Pat. Nos. 8,865,288, 8,886,279, 8,359,083, 7,777,956 and 7,951,300, the contents of which are incorporated herein by reference in their entireties. The implantable electrode array 12 may include features similar to, or otherwise be configured substantially similar to or the same as, the electrode arrays disclosed in one or more of these patents.

As may be best seen in FIG. 2 for example, a handling feature 28 is provided on the first end 22 of the base portion 20. More specifically, the handling feature 28 may be in the form of an elongated fin which extends from the first end 22 of the base portion 20 and transversely to a plane extending between the first end 22 of the base portion 20 and the second end 24 of the base portion 20. The handling feature 28 may, for example, be used to assist in handling and positioning of the implantable electrode array 12 during its implantation. For instance, the handling feature 28 can be used to maneuver the implantable electrode array 12 through bony structures (e.g., a mastoid) and position it to a precise location onto the auditory nerve (or nerve bundle). The handling feature 28 can also provide a way to move the implantable electrode array 12 into certain positions and through certain structures without damaging the implantable electrode array 12 (e.g., damaging the shanks).

Without intending to be limited to any particular configuration, in one form the base portion 20 and the micro-needles 26 may be formed of a material including silicon which is covered in whole or in part by an iridium oxide coating and/or a parylene-C coating. In addition, a portion of the implantable electrode array 12 may include silicone potting deposited thereon, and the handling features 28 may be formed of platinum. However, it should be appreciated that one or more different materials may be employed in addition to or in lieu of the materials herein identified.

The electrical lead 14 is electrically coupled to and extends from the implantable electrode array 12, as indicated above. The electrical lead 14 includes a plurality of wires and one or more of the wires may be individually and electrically coupled with a respective one of the plurality of micro-needles 26. In one form for example, there may be a greater number of the micro-needles 26 than there are number of wires which are electrically coupled to the micro-needles 26. In such a form, not all of the micro-needles 26 of the implantable electrode array 12 may be active. In the illustrated form, the electrical lead 14 includes a first portion 30 and a second portion 32 positioned between the first portion 30 and the implantable electrode array 12. In the first portion 30 of the electrical lead 14, the plurality of wires of the electrical lead 14 may be helically arranged, such as in a helical coil. Further, in the second portion 32 of the electrical lead 14, the plurality of wires of the electrical lead 14 may be straight or linearly arranged. In both the first portion 30 and the second portion 32, the wires of the electrical lead 14 may be over-molded with silicone.

In at least some embodiments, the second portion 32 comprises a bendable portion. As used herein, the term “bendable” refers to a type of wire that exhibits flexible and malleable properties. For example, a bendable portion can include a segment of wire in the second portion 32 that can be deformed in a plastic manner such that the second portion 32 maintains a deformed shape.

In some examples, the second portion 32 of the electrical lead 14 extending away from the implantable electrode array 12 includes a bendable portion that can provide flexibility when positioning the implantable electrode array 12 on a nerve from various angles and at different positions and orientations for proper alignment with the surrounding bone structures and bone grooves. The second portion 32 can include about one millimeters of a bendable portion to 20 millimeters of a bendable portion. In particular examples, the second portion 32 includes about five millimeters of a bendable portion. Such a bendable portion can accommodate a wide variety of anatomical differences in bone structure and orientation of nerves—by bending into a desired bent shape and retaining the bent shape—a discovery made from multiple cadaver experiments and in vivo intraoperative experiments that was not known previously, including the extent of bending needed in the wires and the length of the wires.

The bendable portion of the second portion 32 can include a variety of gauges or thicknesses (e.g., diameters). For example, the bendable portion of the second portion 32 can include a wire in the range of 0.25 mm to 2.0 mm in diameter. In certain implementations, the bendable portion of the second portion 32 is substantially similar in size to an audio nerve (e.g., about 1 mm in diameter). Further, in at least some embodiments, the size or gauge of the bendable portion of the second portion 32 corresponds to a desired degree of flexibility or stiffness. For example, the size or gauge of the bendable portion of the second portion 32 corresponds to an angle of adjustment between zero and 180 degrees. Additionally or alternatively, the size or gauge of the bendable portion of the second portion 32 corresponds to an amount of force to manipulate the bendable portion. For instance, the size or gauge of the bendable portion of the second portion 32 can be smaller to more sensitively manipulate a direction or angle of the bendable portion of the second portion 32 in response to a smaller force load.

The bendable portion of the second portion 32 can include a variety of materials. In at least some examples, the bendable portion comprises one or more wires formed of a ductile, malleable, unreactive, and/or bio-safe material. For instance, the bendable portion comprises one or more platinum wires.

The first portion 30 of the electrical lead 14 also includes a handling feature 34 which, in the illustrated form, is in the form of a handle. More specifically, the handling feature 34 generally includes a fin configuration that couples along a length of the first portion 30 of the electrical lead 14. In one form for example, the handling feature 34 may be formed of silicone, although other variations are possible. The handling feature 34 may, for example, be used to assist in handling and positioning the implantable electrode array 12 and the electrical lead 14 during implantation. For instance, the handling feature 34 can provide improved control when rotating or bending the electrical lead 14 (e.g., with forceps or other surgical instruments).

As indicated above, the auditory signal device 16 may be configured to produce one or more electrical signals representative of observed sound. For example, the auditory signal device 16 may be a subcutaneously implanted stimulator unit that may be in wired or wireless communication with a sound processor located outside the body. The auditory signal device 16 may use radio frequency as wireless communication to the sound processor. The auditory signal device 16 may be further configured to process the received wired or wireless communication from a processor external to the body, and produce one or more electrical signals representative of the observed noises and sounds for communication through the implantable electrode array 12.

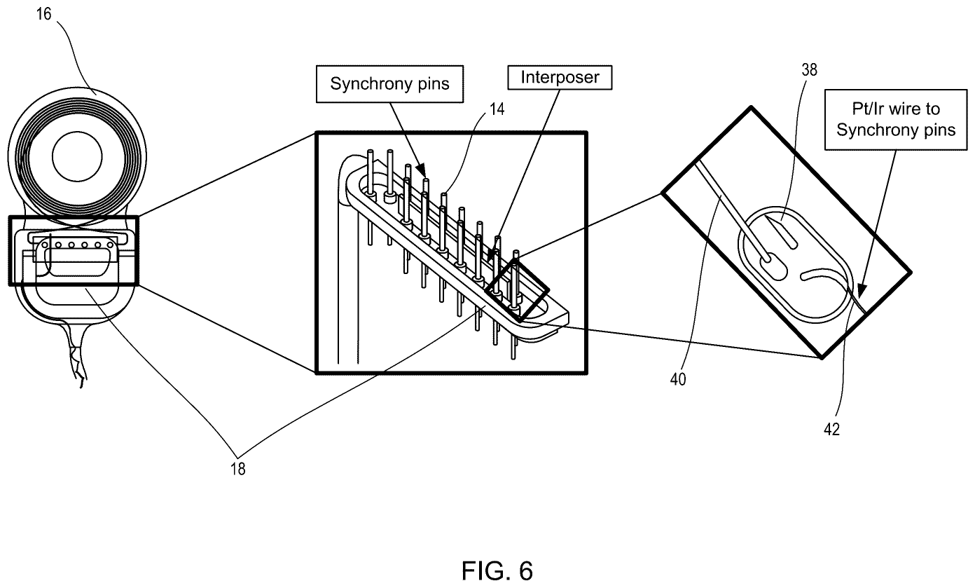

As indicated above, the system 10 also includes an interposer 18 configured to electrically couple the implantable electrode array 12 and the auditory signal device 16 in an arrangement where the one or more electrical signals produced by the auditory signal device 16 may be transmitted through the electrical lead 14 to the implantable electrode array 12. In in one form, the auditory signal device 16 may include a number of electrical pins 36 to and through which the electrical signals produced by auditory signal device 16 may be provided. In this form, the interposer 18 may be configured to engage with the electrical pins of the auditory signal device 16. For example, the interposer 18 may be configured to receive and/or engage with the electrical pins 36 of the auditory signal device 16 as illustrated in FIG. 6 for example, such that the positioning of the interposer 18 relative to the electrical pins 36 may be at least partially fixed.

The interposer 18 includes a number of contact pads 38 with each providing a coupling or bonding location for a respective wire of the electrical lead 14 and a respective wire extending to a respective one of the number of electrical pins 36. By way of example, the number of contact pads 38 provided on the interposer 18 may correspond to the number of electrical pins 36 from which an electrical signal may be provided to the implantable electrode array 12, although variations are possible. FIG. 6 illustrates a wire 40 from the electrical lead 14 and a wire 42 extending to a respective one of the number of electrical pins 36 in connection with one of the contact pads 38.

In one form, the wire 40 (and other wires of the of the electrical lead 14 extending to different respective ones of the contact pads 38) may be formed of a first material or a first combination of materials and the wire 42 (and the other wires extending to the electrical pins 36 from different respective ones of the contacts pads 38) may be formed of a second material or a second combination of materials. Further, each of the contact pads 38 may be formed of a material which is the same as the first material or one of the materials of the first combination of materials or is the same as the second material of the second combination of materials. By way of non-limiting example, the wire 40 may be formed of gold, the wire 42 may be formed of a combination of platinum and iridium, and the contact pads 38 may be formed of platinum. However, other variations in the materials forming the wire 40, the wire 42 and the contacts pads 38 are possible and contemplated. In one form for example, the wires 40 and 42 may be soldered or otherwise coupled to or integrated into a respective contact pad 38 such that the wires 40 and 42 are electrically coupled at the respective contact pad 38. Similarly, an electrical signal provided by the auditory signal device 16 may be provided to the electrical wires of the electrical lead 14 to ultimately produce electrical stimulation by one or more of the micro-needles 26 of the implantable electrode array 12.

In an alternative form, the auditory signal device 16 may include an electrical lead extending therefrom and including a plurality of wires. The interposer 18 may include a number of contact pads 38 each providing a coupling location for a wire of the electrical lead 14 and a wire of the electrical lead extending from the auditory signal device 16. These wires and the contacts pads 38 may be the same or substantially similar to the wires 40 and 42 and contact pads 38 as described in the preceding paragraph, although some variations are also possible. It is also contemplated that in one or more forms, the wires of the electrical lead 14 may be directly electrically coupled to the electrical pins 36 of the auditory signal device 16 or to the wires of the electrical lead extending from the auditory signal device 16. In these forms, it is contemplated that the interposer 18 may be omitted from the system 10.

In one form, the interposer 18 may be formed of a ceramic material, although other variations are possible. For example, forms in which the interposer 18 may be formed of a liquid crystal polymer and include a Ti/Pd interlayer are also possible. In these forms, when the auditory signal device 16 includes the electrical pins 36, the electrical pins 36 may be flattened relative to the interposer 18 and a gold ribbon may be used for a contact pad for electrical coupling between the electrical pins 36 and the wires of the electrical lead 14. In another form, it is contemplated that the interposer 18 could be formed of platinum and include a number of features which are laser cut in order to prevent introduction of foreign materials. In one form when the interposer 18 is formed of platinum, a Pt/Ir foil may be used to weld or solder to the electrical pins 36. In still another form, the interposer 18 may be formed of platinum and aluminum oxide particles.

While not previously described, forms in which the interposer 18 as described herein or otherwise configured may be fitted between the electrical pins 36 of the auditory signal device 16 and accessed from the side for adhering or gluing to the electrical pins 36 are also contemplated. Alternatively, in these forms the interposer 18 may be welded or soldered to the electrical pins 36. In addition, as suggested above, in lieu of the interposer 18, it is also contemplated that the wires of the electrical lead 14 could be coupled directly to one of the electrical pins 36 (when present) or a corresponding wire of an electrical lead extending from the auditory signal device 16. In this form for example, it is contemplated that the electrical pins 36 could be flattened to provide a surface having greater area for coupling a wire of the electrical lead 14. Forms in which the wires are glue bonded and/or weld bonded are also contemplated.

When the interposer 18 electrically couples the implantable electrode array 12 and the auditory signal device 16, or the implantable electrode array 12 and the auditory signal device 16 are otherwise electrically coupled, the one or more electrical signals produced by the auditory signal device 16 may be transmitted through the electrical lead 14 to the implantable electrode array 12. As a result, one or more of the micro-needles 26 may apply a corresponding electrical stimulation to the auditory nerve/auditory nerve bundle in order to create the perception of sound(s) corresponding to that received and recorded by the auditory signal device 16.

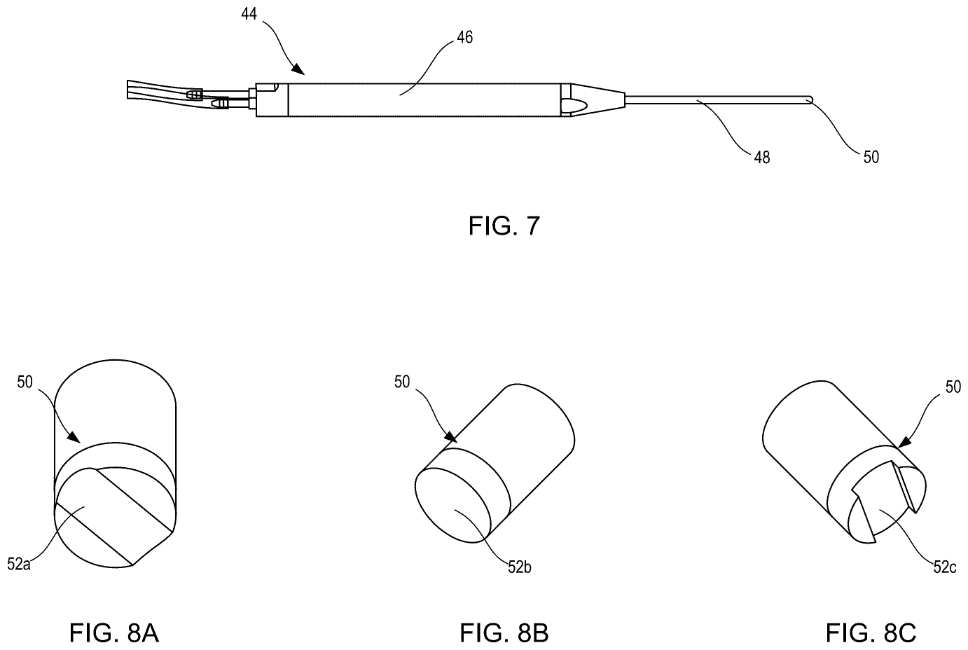

The implantable electrode array 12 may be implanted through any suitable technique (e.g., retrosigmoid (suboccipital) approach, translabyrinthine approach, infralabyrinthine/infracochlear approaches, etc.). In one form for example, the implantable electrode array 12 may be implanted with the device or instrument 44 illustrated in FIG. 7. The instrument 44 includes an outer sleeve or external housing 46 and a plunger member 48 which is positionable and moveable in the external housing 46 to deliver the implantable electrode array 12. The plunger member 48 and the external housing 46 may be sized relative to one another to control insertion velocity of the instrument 44. In one form for example, the length of the plunger member 48 may be increased and the length of the external housing 46 may be decreased in order to decrease the insertion velocity of the instrument 44. The insertion velocity may be controlled by a pneumatic control system that is integrated into the device or instrument 44.

In one embodiment, the plunger member 48 may include a delivery end 50 having a recess formed therein and configured to engage with the implantable electrode array 12. In one form for example, one or more drops of saline may be employed to promote engagement of the implantable electrode array 12 with the delivery end 50 of the instrument 44. In one form, the recess may be formed by a concave slot 52a as illustrated in FIG. 8A. However, in another form, the recess may be formed by a concave dome 52b as illustrated in FIG. 8B or by a rectilinear-shaped slot 52c as illustrated in FIG. 8C. Other variations for the recess formed in the delivery end 50 are also possible.

The electrical lead 14 of the implantable electrode array 12 may be implanted in at least one bone groove created during surgery, which bone groove(s) may retain the electrical lead 14. Such bone groove(s) can help maintain a position of the electrical lead 14 and help provide strain relief. To illustrate, a bone groove of less than one millimeter in diameter and just over one millimeter in depth can permit insertion of the electrical lead 14 therein. Bone grooves of these or other suitable dimensions can be formed with commercially available 0.5-0.8 mm diamond burs. Modest pressure can also help retain the electrical lead 14 within the bone groove.

In one example of bone groove implementation, two grooves can be created within the mastoid cavity. For instance, a first groove can be positioned within the first 1.5 cm of the electrical lead 14 (e.g., at the first portion 30 of the helical bundle) and thus between about 0.5 cm and about 2 cm from the insertion site. In a translabyrinthine approach, this first groove can be achieved posterior and/or inferior to the IAC (interior auditory canal) in the bone posterior and/or inferior to the jugular bulb. In the restrosigmoid approach, a first groove can be formed (e.g., drilled) in the petrous face. A second bone groove can be formed with a more variable position based on the mastoid anatomy (or other bony anatomic variations) and at a position closer to the insertion site of the implantable electrode array 12. In certain implementations, the second bone groove can be placed closer to the cortex, either in the temporal line or the posterior aspect of the mastoid cortical rim.

In at least some examples, a bone groove can be formed relative to an implant body fixed or bedded underneath the temporalis muscle and periosteum. For example, a groove can be formed from the implant bed to the mastoid. In certain implementations, this groove can help avoid exposing the electrical lead 14 or the implantable electrode array 12 to any sharp ledges and/or may provide stability when maneuvering the electrical lead 14 or placing the implantable electrode array 12 into position.

Other stabilizing methods are also herein contemplated. For example, a hemostatic matrix (e.g., Surgicel® absorbable hemostat) can be placed over the implantable electrode array 12 to provide additional surface tension. As another example, abdominal fat strips can be tightly packed against the implantable electrode array 12 to fill the mastoid cavity. In yet another example, the implantable electrode array 12 can be tethered in place via tethering materials, such as a strip of mesh (e.g., polyester Dacron® mesh). For instance, the tethering material can be mounted onto the back of the implantable electrode array 12 and wrapped around one or more nerves (e.g., the auditory nerve) to maintain a position of the implantable electrode array 12.

In one form, a method may include accessing an auditory nerve or auditory nerve bundle and implanting the implantable electrode array 12 in the auditory nerve or auditory nerve bundle. The electrical lead 14 may extend from the implantable electrode array 12 to the auditory signal device 16 which is located at a separate, discrete location on the body of the human being. In one form, the electrical lead 14 extending between the implantable electrode array 12 and the auditory signal device 16 may be placed under the skin of the human being. The auditory signal device 16 may be a subcutaneously implanted stimulator unit that may be in wired or wireless communication with a sound processor located outside the body. The auditory signal device 16 may use radio frequency as wireless communication to the sound processor. The auditory signal device 16 may be further configured to process the received wired or wireless communication from a processor external to the body, and produce one or more electrical signals representative of the received wired or wireless communication through the implantable electrode array 12. The one or more electrical signals may be transmitted to the implantable electrode array 12 and applied to the auditory nerve or auditory nerve bundle through one or more of the micro-needles 26. As a result, the auditory nerve or auditory nerve bundle may be stimulated in a manner intended to create the perception of sound corresponding to the observed sound in the brain of the human being.

The auditory signal device 16 may be used to perform tonotopic mapping of the auditory nerve, by stimulating the auditory nerve using the one or more of the micro-needles 26 at various widths and depths of the auditory nerve, allowing for the activation of a broad frequency range of hearing. The auditory signal device 16 may be used to perform electrical beam steering and or current steering between the one or more of the micro-needles 26. The beam steering and or current steering may enable three dimensional stimulation of the auditory nerve to create higher fidelity perception of sound, whereas traditional cochlear implants are only capable of linear or two dimensional stimulation.

The system 10 may include a sound processor configured to implement a sound coding stimulation strategy. The sound coding stimulation strategy may include a signal processing module, a stimulation module, and a pulse shape module.

The signal processing module of the sound coding stimulation strategy may use a filterbank configured to decompose an acoustic signal into one or more sub-band signals. The filterbank may be implemented through a fast Fourier transform, digital filters, or other types of spectral or temporal filters. For instance, the sound coding stimulation strategy may decompose an acoustic signal into one or more constituent frequencies of that acoustic signal. The signal processing module of the sound coding stimulation strategy may decompose an acoustic signal into any number of sub-bands, and each sub-band may be delivered to the auditory nerve via the same or a reduced number of independent channels of the micro-needles 26. In another example, the signal processing module of the sound coding stimulation strategy may decompose an acoustic signal into 12 possible sub-band frequencies, where each of those 12 possible sub-band frequencies may be delivered independently to the auditory nerve via independent channels of the micro-needles 26 or in some identified arrangement across the auditory nerve based on psychophysical testing and assessment of the induced percepts caused by electrically stimulating each of the micro-needles 26. In an alternative example, the signal processing module of the sound coding stimulation strategy may decompose an acoustic signal into one or more high definition sub-bands, where a subset of the high definition sub-bands may be selectively delivered to the auditory nerve via the system 10.

The stimulation module of the sound coding stimulation strategy may be configured to code the one or more sub-band signals into discrete electrical pulses that may be delivered by the system 10 to the auditory nerve via the micro-needles 26, where each sub-band may be delivered through independent micro-needles 26. As discussed above, the micro-needles 26 may be configured to have varying lengths and therefore varying nerve penetration depths. The penetration depth of the micro-needles 26 may provide access to different nerves in the auditory nerve bundle, where the depth at which each nerve is in the auditory nerve bundle may relate to a sensation of a particular sound frequency.

In one embodiment, the one or more sub-band signals may be categorized by their frequency for delivery by the micro-needles 26 to a specific depth in the auditory nerve that corresponds to the sensation of that sound frequency. In one example, a sub-band of the one or more sub-band signals may be a high frequency sub-band, whereas another sub-band of the one or more sub-band signals may be a low frequency sub-band. In one embodiment, a high frequency sub-band may be delivered by the system 10 to the auditory nerve via the micro-needles 26 using one of the micro-needles that penetrates the auditory nerve at a shallow depth, which is a depth that may be associated with the sensation of high frequency sound. In another embodiment, a low frequency sub-band may be delivered by the system 10 to the auditory nerve via the micro-needles 26 using one of the micro-needles that penetrates the auditory nerve at a deep depth, which is a depth that may be associated with the sensation of low frequency sound.

The stimulation module of the sound coding stimulation strategy may be configured to deliver the sensation of temporal pitch to an auditory nerve via the system 10. For example, the stimulation module of the sound coding stimulation strategy may be configured to provide a stimulation protocol to the auditory nerve via the system 10 that corresponds to a desired temporal pitch and/or loudness of the acoustic signal. In one example, the sensation of temporal pitch could be created by providing electrical stimulation pulses at multiples of zero-crossings of low frequency sub-bands signals of the filterbank. Each channel of the micro-needles 26 of the system 10 may be configured to deliver a different stimulation protocol to the auditory nerve for each channel of the micro-needles 26 to accommodate the different loudness or pitch of each sub-band of the acoustic signal.

The stimulation module of the sound coding stimulation strategy may be configured to stimulate using one or more stimulation coding strategies, including monopolar virtual current steering, bipolar stimulation, tripolar stimulation, or other focused and unfocused stimulation protocols. Each of the one or more stimulation coding strategies may be appropriate for specific stimulation, such as a stimulation intended to produce the sensation of low intensity sound in the auditory nerve. For example, a stimulation coding strategy configured for a focused stimulation protocol may be more appropriate for delivering low intensity sounds, while an unfocused stimulation protocol may be more appropriate for delivering high intensity sounds.

The pulse shape module of the sound coding stimulation strategy may be configured to ensure patient safety during the application of electrical stimulation by the system 10. Both polarities of a stimulation pulse (anodic and cathodic) can depolarize nerve fibers and generate action potentials in the auditory nerve. The pulse shape module may be configured to deliver symmetric, asymmetric pseudo-monophasic, triphasic or other multi-phasic stimulation. The pulse shape module of the sound coding stimulation strategy may be further configured to deliver phase array like stimulation. In such an example, transimpedance matrices (TIM) are used to estimate the voltage distribution caused by each channel of the micro-needles 26. In another example, the pulse shape module of the sound coding stimulation strategy may include the delivery of the inverse of the measured of the simulated neural response which may contain one or more stimulation peaks to reduce the effect of secondary or multiple peaks of excitation. The system 10 may then deliver a stimulation via the micro-needles that is the inverse of the TIM or the neural response to produce a voltage distribution that is a focused as possible.

The stimulation pulses of the implant are generated at a fixed repetition rate and delivered timely interleaved to the electrodes. Observations from previous works with subjects that received an electrical auditory implant consisting of penetrating electrodes in the inferior colliculus (midbrain), the auditory midbrain implant (AMI) showed that stimulating the same electrode at higher rates causes large refractoriness. This problem could be solved by increasing the interpulse interval (gap between two pulses) of stimulation that is equivalent to reduce the repetition rate. However, the necessary reduction does not allow for integration to achieve sufficient loudness perception and modulation of the original signal. A novel stimulation module consists of delivering the signals coming from the same frequency band to multiple electrodes at a higher overall rate achieving sufficient loudness without going into refractoriness. This was originally observed in animal models in which higher activities in the auditory cortex were measured (equivalent to higher loudness). Prerequisite is that the multiple stimulating electrodes elicit similar pitch. That means the stimulated neurons are coherent and being located in the same isofrequency layer. Each electrode itself will be stimulated at lower rates but the overall stimulation rate will be higher than stimulating a single electrode. The effect can be proven by measuring the applied current that is needed to reach equal loudness with multiple electrodes compared to a single electrode. It is possible that the neural patterns elicited by ANI stimulation, which is a more central prosthesis than a CI and therefore more similar to the auditory brainstem implant (ABI) or the auditory midbrain implant, present similar integration properties. In this case the described stimulation mode based on distributing pulses across different electrodes to transmit the same spectral content could improve integration properties.

Typical CI devices may present a constant pulse rate to a given electrode in a specific frequency region of the cochlea, in which the pulse train is amplitude-modulated by the envelope of the bandpass filtered signals for the corresponding frequency range for that cochlear region. The modulation can have varying modulation depths and range of modulation frequencies based on the extracted envelope of the filtered signal for that corresponding frequency channel. As the modulation increases in amplitude, a larger number of nearby neurons are activated in which these neurons correspond to similar or nearby frequencies coded in the cochlea. Thus, the percept becomes louder with a frequency or pitch percept that is still close to the stimulated frequency region of the cochlea.

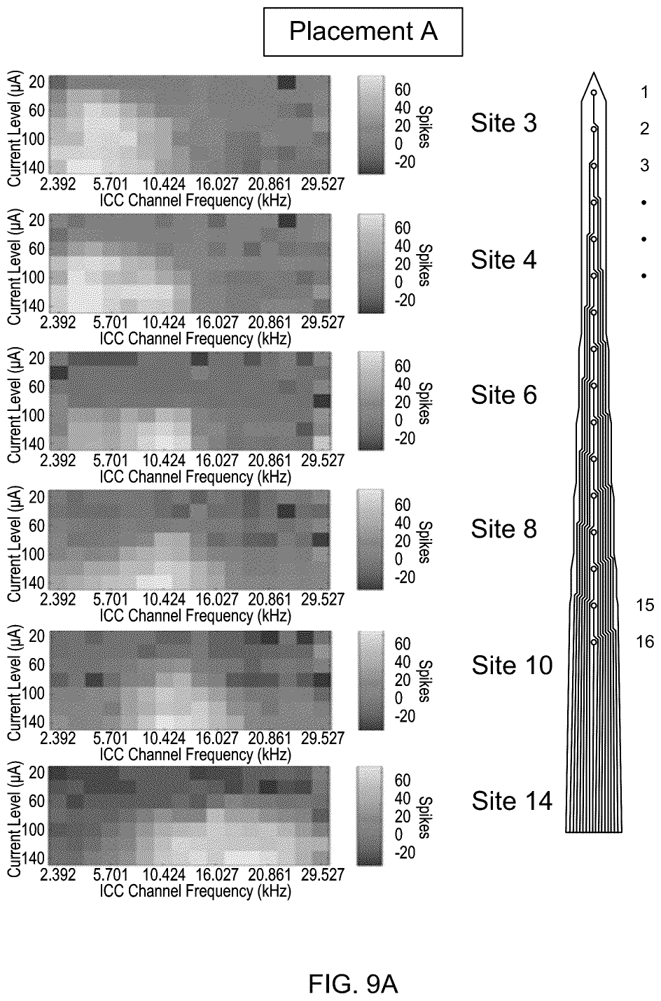

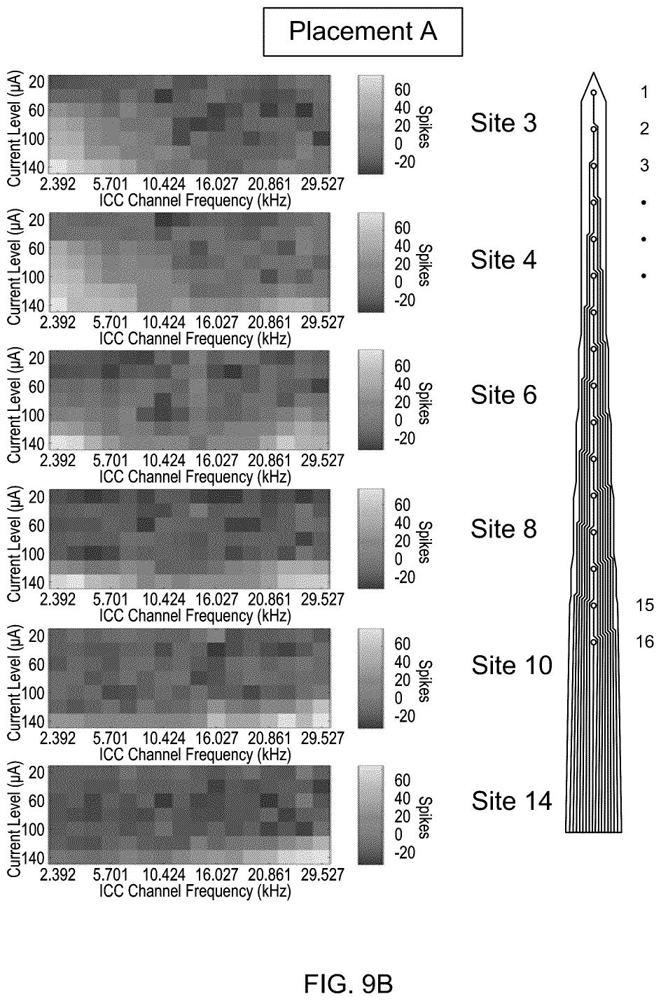

For the auditory nerve, there are nerve fibers that correspond to different frequency percepts, and these fibers can twist en route from the cochlea to the brainstem. As a result, there will be nearby fibers that may not correspond to similar frequencies, but instead have disjointed frequency percepts or frequency and pitch percepts that jump to distant percepts for neighboring activated fibers. We have observed such stimulation patterns in our animal experiments, such as experiments in guinea pigs (see FIGS. 9A and 9B), cats, and monkeys.

In contrast to amplitude modulation stimulation methods implemented in CI devices, the present disclosure includes a stimulation method implementing place modulation stimulation. In other words, rather than increase the current on a given electrode, the presently disclosed stimulation method includes different electrode sites that are activated in a manner that corresponds to similar or nearby frequencies to activate more neurons. Such place-type stimulation can be tuned or optimized by adding more (or altering) activated sites rather than increasing the current on a single electrode. In addition, the electrodes can be mapped to a corresponding frequency or pitch percepts during the fitting process where each electrode is stimulated. In some embodiments, the electrodes are pitch ranked using pitch scaling or pitch ranking psychophysical methods.

In another stimulation strategy method of the present disclosure, a combination of this place modulation and amplitude modulation stimulation strategies can be used. In such an example of place-amplitude modulation stimulation, a given electrode can have its current increased (or decreased) to obtain some range of activated neurons. In addition, other electrodes can be activated corresponding to similar frequencies. These stimulation patterns would be driven by the envelope or bandpass filtered information extracted from the original sound signal. However, instead of using it to amplitude modulate the pulse train to each electrode, there is the addition of stimulating different electrodes to cause louder sensations for similar frequency or pitch percepts. In yet another example, it is possible to additionally (or alternatively) use varying pulse rates for each electrode to increase or modulate the loudness for a similar frequency or pitch percept. Thus, in some embodiments, it is possible to do a combination of a rate modulation, place modulation and/or amplitude modulation across mapped electrodes spanning the auditory nerve with the implantable electrode array of the present disclosure.

Experimental data in support of one or more of the foregoing stimulation strategies is now discussed in relation to FIGS. 9A-9B. In these figures, neural activity patterns in the inferior colliculus central nucleus (ICC) are shown in response to electrical stimulation at various sites of an electrode array implanted in the auditory nerve. A multi-channel electrode used for recording was aligned along the tonotopic axis of the ICC. Each recording channel corresponds to a different frequency region of the ICC, as determined via generation of acoustic tuning curves. For each stimulation site, a stimulus response map (SRM) is generated by presenting a range of current levels (Y-axis) and recording the activity across the frequency channels (X-axis). The color bar for each SRM indicates the number of spikes detected after stimulus delivery. Brighter (yellow) regions on the SRM indicate a higher number of spikes detected while darker (blue) regions denote a lower number of spikes.

From the tip of the stimulating array to the base, Placement A in FIG. 9A shows a gradual shift from low frequency activation (e.g., Sites 3 and 4) to high frequency activation (e.g., Site 14). By contrast, Placement B in FIG. 9B shows a case where the shift from low to high frequency activation is discontinuous. This is especially evident for Sites 5 and 7, which show strong activation of very low and very high frequencies, but little activation of the middle frequencies. Such an activation pattern is likely due to those stimulation sites being placed at a region of the auditory nerve in which groups of low and high frequency fibers run close to one another.

These and/or other embodiments of the present disclosure may be found in Appendices A-H. In particular, Appendices A-H include NIH (National Institutes of Health) Milestone reports, each of which is expressly incorporated herein by reference in their entirety.

In one embodiment, a system includes an implantable electrode array including a plurality of spaced apart micro-needles. The system also includes a first electrical lead electrically coupled to and extending from the implantable electrode array and an auditory signal device configured to produce one or more electrical signals representative of observed sound. An interposer is configured to electrically couple the implantable electrode array and the auditory signal device in an arrangement where one or more electrical signals produced by the auditory signal device may be transmitted through the first electrical lead to the implantable electrode array. In an alternative form, it is contemplated that the first electrical lead may be directly electrically coupled to the auditory signal device without the presence of the interposer.

In one aspect, the interposer is formed of a ceramic material.

In another aspect, the auditory signal device includes a number of electrical pins and the interposer is configured to receive the number of electrical pins.

In one form of this aspect, the interposer includes a number of contact pads and each of the number of contact pads provides a coupling location for a respective wire of the first electrical lead and a respective electrical pin wire which extends to a respective one of the number of electrical pins.

In a further form of this aspect, the respective wire of the electrical lead is formed of a first material or a first combination of materials and the respective electrical pin wire is formed of a second material or a second combination of materials.

In yet a further form of this aspect, each of the number of contact pads is formed of a material which is the same as the first material or one of the materials of the first combination of materials or is the same as the second material or one of the materials of the second combination of materials.

In still a further form of this aspect, the respective wire of the electrical lead is formed of gold, the respective electrical pin wire is formed of a combination of platinum and iridium, and the contact pads are formed of platinum.

In a further aspect, the auditory signal device includes a second electrical lead extending therefrom and the interposer includes a number of contact pads each providing a coupling location for a respective wire of the first electrical lead and a respective wire of the second electrical lead.

In still a further aspect, the first electrical lead includes a plurality of wires and one or more of the plurality of wires is individually and electrically coupled with a respective one of the plurality of micro-needles.

In yet another aspect, the first electrical lead includes a plurality of wires, a first portion and a second portion positioned between the first portion and the implantable electrode array, and in the first portion the plurality of wires are helically arranged and in the second portion the plurality of wires are linearly arranged.

In another aspect, the implantable electrode array extends between a first end and an opposite second end and includes a first handling feature defined by an elongated pin extending transversely from the first end.

In one form of this aspect, the first electrical lead further includes a second handling feature defined by a handle extending from and connecting to the first electrical lead at discrete locations.

In one aspect, the first electrical lead includes a handling feature defined by a handle extending from and connecting to the first electrical lead at discrete locations.

In one aspect, the plurality of micro-needles of the implantable electrode array are arranged in a grid including a number of rows, and a length of each micro-needle in a respective row is different than a length of each micro-needle in a different row.

In another embodiment, an apparatus includes an implantable electrode array including a plurality of spaced apart micro-needles; an electrical lead electrically coupled to and extending from the implantable electrode array and including a plurality of wires; and an interposer including a number of contact pads. One or more of the plurality of wires of the electrical lead are individually and electrically coupled to respective ones of the number of contact pads.

In one aspect, the interposer is formed of a ceramic material.

In another aspect, the plurality of wires of the electrical lead include a first material or a first combination of materials and the number of contact pads include a second material or a second combination of materials.

In one form of this aspect, one or more of the plurality of wires of the electrical lead include gold and the number of contact pads include platinum.

In another aspect, one or more of the plurality of wires is individually and electrically coupled with a respective one of the plurality of micro-needles.

In a further aspect, the electrical lead includes a first portion and a second portion positioned between the first portion and the implantable electrode array, and in the first portion of the plurality of wires are helically arranged and in the second portion the plurality of wires are linearly arranged.

In still a further aspect, the implantable electrode array extends between a first end and an opposite second end and includes a first handling feature defined by an elongated pin extending transversely from the first end.

In one form of this aspect, the electrical lead further includes a second handling feature defined by a handle extending from and connecting to the electrical lead at discrete locations.

In yet another aspect, the electrical lead includes a handling feature defined by a handle extending from and connecting to the first electrical lead at discrete locations.

In one other aspect, the plurality of micro-needles of the implantable electrode array are arranged in a grid including a number of rows and a length of each micro-needle in a respective row is different than a length of each micro-needle in a different row.

In one embodiment, a method includes providing a system which includes:
an implantable electrode array including a plurality of spaced apart micro-needles;
a first electrical lead electrically coupled to and extending from the implantable electrode array; and
an auditory signal device configured to produce one or more electrical signals representative of observed sound.

An interposer is configured to electrically couple the implantable electrode array and the auditory signal device in an arrangement where one or more electrical signals produced by the auditory signal device may be transmitted through the first electrical lead to the implantable electrode array. The method also includes implanting the implantable electrode array in an auditory nerve of a human being, and positioning the auditory signal device at a discrete location on the human being spaced from the implantable electrode array and in electrical communication with the implantable electrode array.

In one aspect, this method further includes providing a device for implanting the implantable electrode array. The device includes an external housing and a plunger member positionable and moveable in the external housing. The plunger member includes a delivery end having a recess formed therein and configured to engage with the implantable electrode array.

In one form of this aspect, the recess is defined by a concave slot, a concave dome, or a rectilinear-shaped slot.

In one aspect, the implantable electrode array is implanted with the device.

It should be appreciated that the subject matter disclosed herein may be used in a variety of different settings. For example, and without limitation, the subject matter disclosed herein may be deployed to assist a subject that suffers from hearing loss. Additionally or alternatively, the subject matter disclosed herein may be used in research or other settings intended to determine the effect of electrical stimulation of the auditory nerve or auditory nerve bundle relative to the creation of the perception of sound.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

The term “substantially” when used in connection with “cylindrical,” “linear,” and/or other geometric relationships is intended to convey that the structure so defined is nominally cylindrical, linear or the like. As one example, a portion of a support member that is described as being “substantially linear” is intended to convey that, although linearity of the portion is desirable, some non-linearity can occur in a “substantially linear” portion. Such non-linearity can result from manufacturing tolerances, or other practical considerations (such as, for example, the pressure or force applied to the support member). Thus, a geometric construction modified by the term “substantially” includes such geometric properties within a tolerance of plus or minus 5% of the stated geometric construction. For example, a “substantially linear” portion is a portion that defines an axis or center line that is within plus or minus 5% of being linear.

As used herein, the term “set” and “plurality” can refer to multiple features or a singular feature with multiple parts. For example, when referring to a set of electrodes, the set of electrodes can be considered as one electrode with multiple portions, or the set of electrodes can be considered as multiple, distinct electrodes. Additionally, for example, when referring to a plurality of electrochemical cells, the plurality of electrochemical cells can be considered as multiple, distinct electrochemical cells or as one electrochemical cell with multiple portions. Thus, a set of portions or a plurality of portions may include multiple portions that are either continuous or discontinuous from each other. A plurality of particles or a plurality of materials can also be fabricated from multiple items that are produced separately and are later joined together (e.g., via mixing, an adhesive, or any suitable method).

What is claimed is:

1. An auditory nerve stimulation system, comprising:
an implantable electrode array;
a first electrical lead electrically coupled to and extending from the implantable electrode array;
an auditory signal device configured to produce one or more electrical signals representative of communications received from an external processor; and
an interposer electrically coupled to the first electrical lead and the auditory signal device to enable one or more electrical signals produced by the auditory signal device to be transmitted through the first electrical lead to the implantable electrode array.

2. The auditory nerve stimulation system of claim 1, wherein the interposer is formed of a ceramic material.

3. The auditory nerve stimulation system of claim 1, wherein the auditory signal device includes electrical pins and the interposer is configured to receive and electrically couple with the electrical pins.

4. The auditory nerve stimulation system of claim 3, wherein the interposer includes contact pads and each of the contact pads provides a coupling location for a respective wire of the first electrical lead and a respective electrical pin wire which extends to a respective one of the electrical pins.

5. The auditory nerve stimulation system of claim 4, wherein the respective wire of the first electrical lead is formed of a first material or a first combination of materials and the respective electrical pin wire is formed of a second material or a second combination of materials.

6. The auditory nerve stimulation system of claim 5, wherein each of the contact pads is formed of a material which is the same as the first material or one of the materials of the first combination of materials or is the same as the second material or one of the materials of the second combination of materials.

7. The auditory nerve stimulation system of claim 6, wherein the respective wire of the first electrical lead is formed of gold, the respective electrical pin wire is formed of a combination of platinum and iridium, and the contact pads are formed of platinum.

8. The auditory nerve stimulation system of claim 1, wherein the auditory signal device includes a second electrical lead extending therefrom and the interposer includes contact pads each providing a coupling location for a respective wire of the first electrical lead and a respective wire of the second electrical lead.

9. The auditory nerve stimulation system of claim 1, wherein the first electrical lead includes a plurality of wires and one or more of the plurality of wires is individually and electrically coupled with a respective one of the plurality of micro-needles.

10. The auditory nerve stimulation system of claim 1, wherein the first electrical lead includes a plurality of wires, a first portion and a second portion positioned between the first portion and the implantable electrode array, and wherein in the first portion the plurality of wires are helically arranged and in the second portion the plurality of wires are linearly arranged.

11. The auditory nerve stimulation system of claim 10, wherein the second portion comprises a bendable portion.

12. The auditory nerve stimulation system of claim 11, wherein the bendable portion comprises one or more platinum wires.

13. The auditory nerve stimulation system of claim 1, wherein the implantable electrode array extends between a first end and an opposite second end and includes a first handling feature defined by an elongated pin extending transversely from the first end.

14. The auditory nerve stimulation system of claim 13, wherein the first electrical lead further includes a second handling feature defined by a handle extending from and connecting to the first electrical lead at discrete locations.

15. The auditory nerve stimulation system of claim 1, wherein the electrode array includes a plurality of micro-needles, where the plurality of micro-needles of the implantable electrode array are arranged in a grid including a number of rows, and wherein a length of each micro-needle in a respective row is different than a length of each micro-needle in a different row.

16. The auditory nerve stimulation system of claim 15, wherein the length of each micro-needle of the plurality of micro-needles ranges from about 0.1 millimeters to about 1.2 millimeters.

17. An auditory nerve stimulation apparatus, comprising:
an implantable electrode array including a plurality of spaced apart micro-needles;
an electrical lead electrically coupled to and extending from the implantable electrode array and including a plurality of wires; and
an interposer including contact pads;
wherein one or more of the plurality of wires of the electrical lead are individually and electrically coupled to respective ones of the contact pads.

18. The auditory nerve stimulation apparatus of claim 17, wherein the interposer is formed of a ceramic material.

19. The auditory nerve stimulation apparatus of claim 17, wherein the plurality of wires of the electrical lead include a first material or a first combination of materials and the number of contact pads include a second material or a second combination of materials.

20. The auditory nerve stimulation apparatus of claim 19, wherein one or more of the plurality of wires of the electrical lead include gold and the number of contact pads include platinum.

21. The auditory nerve stimulation apparatus of claim 17, wherein one or more of the plurality of wires is individually and electrically coupled with a respective one of the plurality of micro-needles.

22. The auditory nerve stimulation apparatus of claim 17, wherein the electrical lead includes a first portion and a second portion positioned between the first portion and the implantable electrode array, and wherein in the first portion the plurality of wires are helically arranged and in the second portion the plurality of wires are linearly arranged.

23. The auditory nerve stimulation apparatus of claim 17, wherein the implantable electrode array extends between a first end and an opposite second end and includes a first handling feature defined by an elongated pin extending transversely from the first end.

24. The auditory nerve stimulation apparatus of claim 23, wherein the electrical lead further includes a second handling feature defined by a handle extending from and connecting to the electrical lead at discrete locations.

25. The auditory nerve stimulation apparatus of claim 17, wherein the plurality of micro-needles of the implantable electrode array are arranged in a grid including a number of rows and a length of each micro-needle in a respective row is different than a length of each micro-needle in a different row.

26. A method, comprising:
providing a system according to claim 1;
implanting the implantable electrode array in an auditory nerve of a human being; and
positioning the auditory signal device at a discrete location on the human being spaced from the implantable electrode array and in electrical communication with the implantable electrode array.

27. The method of claim 26, further comprising providing a device for implanting the implantable electrode array, the device including an external housing and a plunger member positionable and moveable in the external housing, and the plunger member including a delivery end having a recess formed therein and configured to engage with the implantable electrode array.

28. The method of claim 27, wherein the recess is defined by a concave slot, a concave dome, or a rectilinear-shaped slot.

29. The method of claim 27, wherein the implantable electrode array is implanted with the device.

30. The method of claim 26, wherein the implantable electrode array comprises:
a first set of electrode shanks; and
a second set of electrode shanks shorter than the first set of electrode shanks.

31. The method of claim 30, wherein:
the implantable electrode array is oriented with the first set of electrode shanks positioned closer than the second set of electrode shanks to a cochlea of the human being; and
the implantable electrode array is oriented with the second set of electrode shanks positioned closer than the first set of electrode shanks to a brain stem of the human being.

32. The method of claim 26, further comprising:
forming at least one bone groove within a bone structure of the human being; and
positioning, within the bone structure, an electrical lead coupled to the implantable electrode array.

33. A method comprising:
providing a system for stimulating neural tissue, the system comprising:
an implantable electrode array;
a first electrical lead electrically coupled to and extending from the implantable electrode array;
an auditory signal device configured to produce one or more electrical signals representative of communications received from an external processor; and
an interposer configured to electrically couple the implantable electrode array and the auditory signal device in an arrangement where one or more electrical signals produced by the auditory signal device may be transmitted through the first electrical lead to the implantable electrode array; and
transmitting place-amplitude stimulation signals from the system to the neural tissue at a plurality of electrodes sites corresponding to the implantable electrode array.

34. The method of claim 33, wherein the implantable electrode array comprises electrodes that are pitch ranked.

35. The method of claim 33, where transmitting place-amplitude stimulation signals comprises:
transmitting a first signal at a first frequency via a first electrode of the implantable electrode array; and
transmitting a second signal at a second frequency via a second electrode of the implantable electrode array, the second frequency differing from the first frequency.

36. The method of claim 33, further comprising transmitting at least one of amplitude-modulated stimulation signals or rate-modulated signals from the system to the neural tissue at the plurality of electrodes sites corresponding to the implantable electrode array.
